(19) United States
(12) Patent Application Publication
(10) Pub. No.: US 2022/0375563 A1
(43) Pub. Date: Nov. 24, 2022

(54) METHOD AND APPARATUS FOR INDIVIDUALIZED ADMINISTRATION OF MEDICAMENTS FOR ENHANCED SAFE DELIVERY WITHIN A THERAPEUTIC RANGE

(71) Applicant: UNIVERSITY OF MARYLAND, BALTIMORE, BALTIMORE, MD (US)

(72) Inventors: Christopher Vincent Rackauckas, Cambridge, MA (US); Vijay Vaturi, Centreville, VA (US)

(21) Appl. No.: 17/753,294

(22) PCT Filed: Aug. 26, 2020
(86) PCT No.: PCT/US20/47889

§ 371 Date: Feb. 25, 2022

Related U.S. Application Data
(60) Provisional application No. 62/891,602, filed on Aug. 26, 2019.

(51) Int. Cl. G16H 20/10; G16H 10/60

(52) U.S. Cl. CPC G16H 20/10; G16H 10/60

(57) ABSTRACT

Techniques for generating a dosing protocol for an individual include receiving first and second and third data. First data indicates, for dose response to a medicament, a continuous multivariate model with at least one distribution parameter characterizing variations in the population. Second data indicates a therapeutic range of values for the dose response. Third data indicates a cost function based on distance of a dose response from the therapeutic range. The method includes evaluating for a candidate dose an expected cost based at least in part on the distribution parameter and a Koopman transform of the cost function. When the expected cost is less than a threshold cost, the candidate dose of the medicament is administered to a subject.

### CROSS-REFERENCE TO RELATED APPLICATIONS

This application claims benefit of Provisional Appln. 62/891,602, filed Aug. 26, 2019, the entire contents of which are hereby incorporated by reference as if fully set forth herein, under 35 U.S.C. § 119(e).

### BACKGROUND

Different patients process administered drugs differently; and for some drugs or for some neonates, or some combination, the variability in delivery of drug concentration for the same amount administered can have a strong bearing on the effectiveness of the drug. As used herein, the term medicament means any material administered to a subject for therapeutic effect, including drugs and other biological agents, such as large molecules, nucleic acids, viruses and bacteria. In general, one can say that, for some medicaments, careful and precise management of administration and delivery is of critical importance. The process of managing administration and delivery of drugs has been called therapeutic drug management (TDM) in the health care industry. Here we use the term TDM to refer to the management of both drugs and other medicaments.

If the medicament is delivered in too low a dose, the efficacy of the treatment may be compromised; while, if administered in too a high a dose deleterious and even dangerous side effects may occur.

### SUMMARY

Techniques are provided for more efficiently determining proper dosing of medicaments during individualized treatment of a subject, which dosing has increased chance to avoid costly excursions from a target therapeutic range.

In a first set of embodiments, a method executed automatically on a processor for generating a dosing protocol for an individual, includes receiving first data that indicates, for dose response to a medicament, a continuous multivariate model with at least one distribution parameter characterizing variations in the population. The method also includes receiving second data that indicates a therapeutic range of values for the dose response. The method further includes receiving third data that indicates a cost function based on distance of a dose response from the therapeutic range. Still further, the method includes evaluating for a candidate dose an expected cost based at least in part on the distribution parameter and a Koopman transform of the cost function, Yet further, the method includes, when the expected cost is less than a threshold cost, causing the candidate dose of the medicament to be administered to a subject.

In various embodiments of the first set, said evaluating the expected cost further comprising performing multidimensional quadrature integration.

In some embodiments of the first set, the at least one distribution parameter is based on a distribution of values in electronic health records (EHR).

In some embodiments of the first set, the medicament is a type selected from a group comprising anti-epileptics, anti-coagulants, anti-arrhythmics, and immunosuppressants.

In some embodiments of the first set, the distance of a dose response from the therapeutic range for the cost function is indicated by a therapeutic index.

In other sets of embodiments, a computer-readable medium or a system is configured to perform one or more steps of one or more of the above methods.

Still other aspects, features, and advantages are readily apparent from the following detailed description, simply by illustrating a number of particular embodiments and implementations, including the best mode contemplated for carrying out the invention. Other embodiments are also capable of other and different features and advantages, and its several details can be modified in various obvious respects, all without departing from the spirit and scope of the invention. Accordingly, the drawings and description are to be regarded as illustrative in nature, and not as restrictive.

### BRIEF DESCRIPTION OF THE DRAWINGS

Embodiments are illustrated by way of example, and not by way of limitation, in the figures of the accompanying drawings in which like reference numerals refer to similar elements and in which:

FIG. 1 is a block diagram that illustrates an example pharmacokinetic and pharmacodynamics system model, used according to an embodiment;

FIG. 2A is a pair of graphs that illustrate an example relationship between initial conditions probability density function (pdf) and resulting dynamic variable probability density (pdf) function, respectively, according to an embodiment;

FIG. 2B is a set of plots that illustrates interaction of uncertainty in initial conditions represented by a pdf and a cost function using a Koopman adjunct process, according to an embodiment;

FIG. 3 is flow diagram that illustrates an example method for objectively and automatically determining dosing regimen, according to an embodiment;

FIG. 4 is a graph that illustrates example convergence of the Koopman Expectation calculation compared to a previous method using Monte Carlo Expectation, according to an example embodiment;

FIG. 5 is a block diagram that illustrates a computer system upon which an embodiment of the invention may be implemented; and,

FIG. 6 illustrates a chip set upon which an embodiment of the invention may be implemented.

### DETAILED DESCRIPTION

A method and apparatus are described for determining, during treatment of a subject, individualized protocols for administering medicaments, which have increased chance to be maintained in the subject within a therapeutic range. In the following description, for the purposes of explanation, numerous specific details are set forth in order to provide a thorough understanding of the present invention. It will be apparent, however, to one skilled in the art that the present invention may be practiced without these specific details. In other instances, well-known structures and devices are shown in block diagram form in order to avoid unnecessarily obscuring the present invention.

Notwithstanding that the numerical ranges and parameters setting forth the broad scope are approximations, the numerical values set forth in specific non-limiting examples are reported as precisely as possible. Any numerical value, however, inherently contains certain errors necessarily resulting from the standard deviation found in their respective testing measurements at the time of this writing. Furthermore, unless otherwise clear from the context, a numerical value presented herein has an implied precision given by the least significant digit. Thus, a value 1.1 implies a value from 1.05 to 1.15. The term “about” is used to indicate a broader range centered on the given value, and unless otherwise clear from the context implies a broader range around the least significant digit, such as “about 1.1” implies a range from 1.0 to 1.2. If the least significant digit is unclear, then the term “about” implies a factor of two, e.g., “about X” implies a value in the range from 0.5X to 2X, for example, about 100 implies a value in a range from 50 to 200. Moreover, all ranges disclosed herein are to be understood to encompass any and all sub-ranges subsumed therein. For example, a range of “less than 10” for a positive only parameter can include any and all sub-ranges between (and including) the minimum value of zero and the maximum value of 10, that is, any and all sub-ranges having a minimum value of equal to or greater than zero and a maximum value of equal to or less than 10, e.g., 1 to 4.

In various embodiments, a corresponding precision therapeutics toolset would be directly applicable to all medicaments that benefit from TDM. Prediction models other than those for precision dosing can be incorporated, e.g., to assess appropriateness of palliative chemotherapy in patients with metastatic cancer with dismal prognosis by predicting survival; to use appropriate anti-diabetic therapy in patients with type 2 diabetes of varied pathophysiology; or to predict cardiovascular event risk in a given time period.

### 1. OVERVIEW

Biologically and pharmacologically plausible systems models such as Pharmacokinetic (PK) and Pharmacodynamic (PD) (PK/PD) and Physiologically-Based Pharmacokinetic (PBPK) models are tailored for concentration levels of a dose in individual subjects. The systems models for precision therapeutics are highly non-linear and require multiple levels of uncertainty and variation, leading to the use of non-linear mixed effects (NLME) models. NLME for statistical inference is demonstrated in a large literature of applied statistics. Moreover, the form of NLME models limits the types of usable patient data to easily provided quantities such as age, sex, and weight. The systems models proposed here with NLME may be used in conjunction with modern machine learning algorithms such as deep (convolutional) neural networks, density-based clustering on diffusion mapped data, or gradient-boosted regression trees to determine values of model parameters of various categories. This pushes well beyond modeling for the averages to a space filled with complex multi-dimensional healthcare information with both fixed and random effects models. Such modeling attempts to capture a therapeutically significant subset of the underlying complex pharmacological mechanisms that can be used to individualize therapeutics one patient at a time. As used herein, a subject is a human or animal patient or healthy recruit or surrogate or a simulation of same.

### 1.1 Non-Linear Mixed Effects (NLME) Models Background

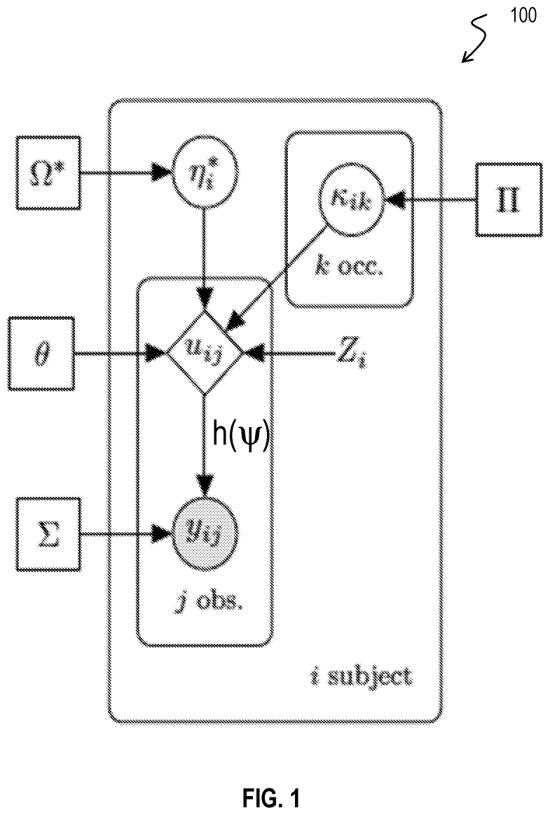

FIG. 1 is a block diagram that illustrates an example pharmacokinetic and pharmacodynamics system model 100, used according to an embodiment. In FIG. 1, the rectangle nodes denote variable parameters, circles denote random quantities which are either latent (unfilled) or observed (filled), diamonds are deterministic given the inputs, and nodes without a border are constant. The system model 100 describes the evolution of one or more observable medicament levels yᵢⱼ for individual i at observation time elements j. Such a system is defined by three components: random effects; dynamics; and observations.

An individual's random effect, represented by the symbol ηᵢ, captures the inter-individual variability (IIV) for individual i within a population of subjects. The variable ηᵢ, represents one or more random effects for individual i and thus, in general, is a vector. The distribution of this effect in the whole population is parametrized by Ω. Sometimes, one captures other individual random effects, such as the inter-occasion variability (IOV) effect denoted by [symbol] ᵢₖ at each occasion k for individual i, e.g., when an individual patient visits a hospital on multiple occasions for a tissue sample to determine medicament levels (such as a blood draw to determine drug concentration value in the blood at those times). [symbol] ᵢₖ represents one or more inter-occasion random effects for individual i and thus, in general, is a vector. The distribution of the inter-occasion effect over the whole population is denoted by Π. The most common choice for random effect components is some sort of multivariate normal distribution N with zero mean of the form ηᵢ=N(0, Ω) and [symbol] ᵢₖ=N(0, Π).

As used herein, a distribution parameter characterizes variations in the population regardless of origin, e.g., variation among individuals, such as those based on covariates, inter-event variability for an individual, distribution around a population mean of pooled data, and uncertainty in measurements, among others. For example, after doing a likelihood estimation in something like a Bayesian sense, one learns a distribution for ηᵢ in terms of Ω, but one also might get a distribution for the θ terms. The maximum likelihood estimation (approximately) returns the maximum of this distribution; but, Bayesian fitting methods don't just give one point back but instead a point with uncertainty (similar to an estimate with error bars). So one can have uncertainty in the θ terms by incorporating those error bars. These errors can come from many sources: measurement error in the data, numerical error in the nonlinear routines, etc. Also, in some embodiments, one does not do individual-specific estimations, but still wants to do optimal dosing under the uncertainty with respect to the estimated error in the θ fits.

Dynamics control the time evolution of the system. A dynamic variable uᵢⱼ represents the unobserved (latent) dynamics of the model that unfold at a sequence of time points tᵢⱼ for individual i and time element j. uᵢⱼ represents one or more latent dynamic variables for individual i and thus, in general, is a vector. In some embodiments, these dynamics are provided as the solution to a system of differential equations including as parameters one or more nonrandom parameters in a vector θ, the one or more random effects ηᵢ, and zero or more subject-specific values for covariates variables in vector Zᵢ. Co-variates Zᵢ is a vector of zero or more observable characteristics of individual i, which are known to vary with yᵢⱼ that is being modeled. In other embodiments, the dynamics are modeled in other ways, such as using continuous and discrete Markov chains, or stochastic, delay, hybrid, and differential-algebraic variants of the differential equations. In some embodiments, there are no random effects represented by ηᵢ, like in first dose estimation.

The functional relationship among these variables is expressed in Equation 1a.

uᵢⱼ=uᵢ(tᵢⱼ)=u(tᵢⱼ;θ,ηᵢ;Zᵢ) (1a)

In response to a dose Dᵢ given to subject i, the dynamic variable vector uᵢ changes with time, designated by the time derivative \dot{u}ₗ, as given by Equation 1b.

\dot{u}ₗ=f(uᵢ,Dᵢ,ηᵢ,θ,Zᵢ) (1b)

Because the mean of the distribution N(0, Ω) is zero, the expected value of ηᵢ=0 and the expected patient outcome, given only the prior known information Zi, is the dynamics predicted by θ with ηᵢ=0.

The actual differential equations represented by Equation 1b have a form depending on the medicament being modeled.

Observations yᵢⱼ is a vector of one or more actual observed response variables (also called dependent variables). These are related to the dynamic variables uᵢⱼ by an observation function ψ; and, assumed to have some distribution which depends on uᵢⱼ and zero or more additional random parameters, such as random errors, represented by the vector σ whose distribution over the whole population is denoted by Σ. as given by Equation 1b.

yᵢⱼ=h(ψ(uᵢⱼ,Zᵢ,ηᵢ,θ,σ)) (1c)

A nonlinear mixed effect (NLME) model output is determined as follows. From the distributions (Ω, Π, Σ) the random variables (ηᵢ, [symbol] ᵢₖ, σ) are defined. The covariates Zᵢ for each individual are given, as are the parameter values θ for the population. These pieces then define the parameters for the dynamical model for a particular set of differential equations represented by Equation 1b, which is solved (analytically or numerically). Then the observation function ψ is applied, then the prediction model h with error values ϵᵢ (having standard deviation σ) is applied as represented in Equation 1c, resulting in modeled observations, e.g., for a simulation. As a result, the modeling process can be thought of as the map from inputs to outputs given by an expression labeled Equation 1d.

(θ,Zᵢ,Ω,Π,Σ)→yᵢⱼ=yᵢ(tᵢⱼ) (1d)

For training and for individualized administration of a medicament, the interest is in the inverse problem of learning more about the model given data of real observations, represented by the symbol dᵢⱼ=dᵢ (tᵢⱼ). The desire is to learn one or more of θ; Zᵢ, and Ω, Π, Σ, (thus effectively the amount of randomness for the random effects, random occasions and random measurements) or more explicitly at least random draws from those last three distributions ηᵢ, [symbol] ᵢₖ, and ϵᵢ. This inverse problem, summarized as finding the parameters such that ∥d−y∥ is minimized, is known as the estimation problem. This minimization is performed by finding the parameters that maximize the population likelihood or via Maximum A Posteriori (MAP) estimation. Common methods which approximate the population likelihood include: 1) First order conditional estimation 2) Laplace approximations 3) Expectation maximization (sampling-based methods). In addition, more accurate posterior simulation algorithms such as Hamiltonian Monte Carlo, Stochastic Approximation Expectation-Maximization (SAEM), and Approximate Bayesian Computation (ABC) are used for parameter estimation in some embodiments. In prior work machine learning, such as neural networks, are also used to determine the parameters that define the deterministic, dynamic and random elements of the design model.

In PK compartment models, drug concentrations (C) in the compartments are equal to the amounts (A) divided by volumes (V). For two compartments, C1=A1/V1 and C2=A2/V2. Drug concentration in the central compartment (C1) is equal to the concentration in the plasma (CP). Clearance (CL, in units of liters, L, per hour) is often used instead of the fractional rate constants kₐ (in units of per hour, not to be confused with occurrence k). In pharmacokinetics, the distribution volume is given in volume units (L), and rate constants can be represented as the ratio of clearance and distribution volume, e.g., kₐ=CL/V. In the case of oral administration of the drug, at time t=0 the amount of drug in the central and peripheral compartments is zero (A1(0)=A2(0)=0), and the initial amount in gastrointestinal tract (effective dose) is: AGI(0)=D×S×F, where D is the administered dose of the drug, S is the salt factor (fraction of administered dose that is made up of pure drug), and F is the bioavailability factor (fraction of dose that reaches the systemic circulation). Parameters often used in PK models include D=dose, τ=dosing interval, CL=clearance, Vd=volume of distribution, kₑ=elimination rate constant, kₐ=absorption rate constant, F=fraction absorbed (bioavailability), K₀=infusion rate, T=duration of infusion, and C=plasma concentration.

In example embodiments, one or more of these parameters might be described or affected by a distribution about a typical value of random values for different individuals.

Electronic health records (EHR) provide a rich dataset for employing machine learning methods. Thus, in various embodiments, numerical solutions for the differential equations of a NLME model are implemented as an activation layer of a neural net architecture. By directly encoding the ODE dynamics and events into the neural net layer via its activation function, one can retain the properties of the modeling-based approach so that the outputted values (the predicted concentrations) are biologically realistic and follow the proposed clinical model. The population parameters and random distribution function are learned during this training, while maintaining the ODE relationships. In some embodiments, this dynamic layer is then embedded in an end to end neural network that has as input values and any measurements of therapeutic effects on an individual; and; has as an output layer the expected dose response and confidence in (probability of) the same. In other embodiments, other methods of determining the model θ parameters are used.

The parameters of this system would then be the input nodes of the dynamical layer, which are chained from earlier layers of a neural network as predictions from arbitrary datasets. In this way, the relationship between electronic health records (EHR) records, imaging results, and molecular biological assays (protein assays, SNP arrays, RNA-seq) can be trained and deduced programmatically without requiring a modeler assumption; directly generalizing previous PK/PD modeling approaches to contemporary big data.

For example, in a Bayesian estimation process, given observations yᵢ of the underlying dynamical system uᵢ for multiple individuals i, and given the likelihood of given fixed effects θ, and given the random effects variance Ω, the population likelihood L is related to the individual likelihoods Lᵢ by Equation 1e.

L(Ω,θ,y)=ΠᵢLᵢ(uᵢ,yᵢ) (1e)

Here, Π indicates the product of the individual likelihoods (as distinguished from distribution Π in FIG. 1). Lᵢ is the likelihood for individual i which is a chosen functional form; and, For example, a common choice of Lᵢ is the normal distribution centered around the data with measurement error variance σ, such as given by Equation 1f.

Lᵢ˜N(uᵢ−yᵢ,σ) (1f)

The parameters θ and ηᵢ are chosen to maximize the likelihood of the observation, which is equivalent to choosing the parameter values that minimize ∥uᵢ−yᵢ∥. In this Bayesian estimation procedure, it is desired to capture the uncertainty in the parameters θ and ηᵢ instead of performing a deterministic procedure, like maximum likelihood. In the Bayesian approach, the posterior probabilities distributions for θ and ηᵢ are derived based on assumed prior distributions. This is done iteratively starting with an initial distribution, such as a normal distribution.

Common probabilistic programming languages, such as Stan or Turing, can be utilized to determine these posterior distributions using Markov Chain Monte Carlo (MCMC) techniques such as Hamiltonian Monte Carlo. Plots of estimated distributions of NLME parameters computed using the Pumas and other software via HMC against data, showcase how the discrete MCMC chains can be reinterpreted as probability distributions via a kernel density estimate (KDE).

### 1.2 Method

Once the model parameters and their uncertainties are determined, the model is used to determine the dosing regimen for an individual. The dosing regimen specifies how much, where and when to administer the medicament to the subject. As used herein a “dose” refers to an amount of a medication that is administered to a position inside the subject at a particular time, rather than the resulting concentration level in a target tissue of the subject. Because of the random components describing variations among individuals and variations in results between events of a single individual, any particular dose can lead to a range of possible outcomes in terms of actual observed medicament concentrations. An understanding of that range of possible results should inform the decision on the original dose. In many prior approaches, the expected result is expressed in terms of the probability of the actual concentration occurring in a therapeutic range. If that probability is high enough for a particular dose, then that dose is considered acceptable.

It is noted here that, of all acceptable doses, a subset may be preferable because the probability of deleterious deviations from the therapeutic range are smaller than for other administered doses in the acceptable range. Therefore, in various embodiments, a cost function is defined that includes a measure of weighted deviations from the therapeutic range; and the dose determined is based on reducing or minimizing the weighted deviations. This formulation reduces or minimizes a cost, where the cost is based on the probability of high excursions of the subject's drug response from known safety windows. While this cost function can result in high values, it is a better indication of a true cost to the subject; and, thus; is more useful for optimizing dose (compared to minimizing the distance from some optimal response curve, which does not capture the fact that any solution within the safety window is “good”). Thus, the quantity of interest in some embodiments, is the probability of rare but large excursions.

In some embodiments, this cost is evaluated based on using a Koopman adjoint approach to determine the distribution of initial conditions, e.g., dose, which approach is several times more efficient than a naïve Monte Carlo approach to determining that distribution. In some embodiments, increased efficiency is achieved by employing graphical processing unit (GPU) accelerated data-parallel numerical solutions to ordinary differential equations (ODEs), in addition to the Koopman adjoint approach.

It is assumed that Bayesian estimation or prior knowledge or other technique, such as maximum likelihood, has given sufficient estimates of posterior distributions for θ and ηᵢ and the following question is to be answered: what is an optimal or advantageous dosage regimen Dᵢ for patient i? The rest of this section is focused on this single patient i; and, thus the subscript i will be dropped for this section.

To describe a typical approach, the Frobenius-Perron (FP) operator is introduced. Define S(x)=u(t) where u(0)=x. In other words, S(x) is the solution of the dynamical system where the initial condition is x and tis time. To compute probabilistic statements on patient outcomes with respect to uncertainty of the initial condition the FP operator is used. First, it is noted that uncertainties in parameters (η and θ) are incorporated into this calculation. For the ordinary differential equations, the extended system of ODE is expressed by Equation 1b (repeated below as Equation 2), Equation 3, and Equation 4.

\dot{u}ₗ=f(uᵢ,Dᵢ,ηᵢ,θ,Zᵢ) (2)

\dot{η}=0 (3)

\dot{θ}=0 (4)

Here, η(0)=η and θ(0)=θ, where the argument is time and 0 indicates initial time. Thus, the parametric uncertainties can be treated as uncertainties in the initial condition on this extended system. Furthermore, without loss of generality, the uncertainty is modeled via the initial conditions in a probability space ( [symbol] ⁿ, [symbol] , μ) where [symbol] ⁿ is the set of real numbers. As known in formal mathematics measure theory, this is a type of measure space. In probabilistic terms, [symbol] is an event set and p is a measure on the event set that defines the probability for each event.

For a given dynamical system S: [symbol] ⁿ→ [symbol] ⁿ, its associated FP operator, P_{S}, is defined such that Equation 5 is satisfied.

∫_{A}P_{S}f(x)μ(dx)=∫_{S}⁻¹_{(A)}f(x)μ(dX),∀A∈ [symbol] (5)

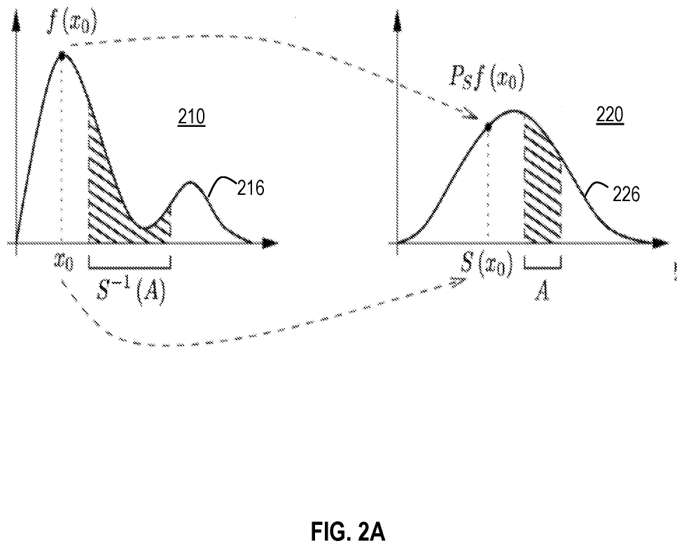

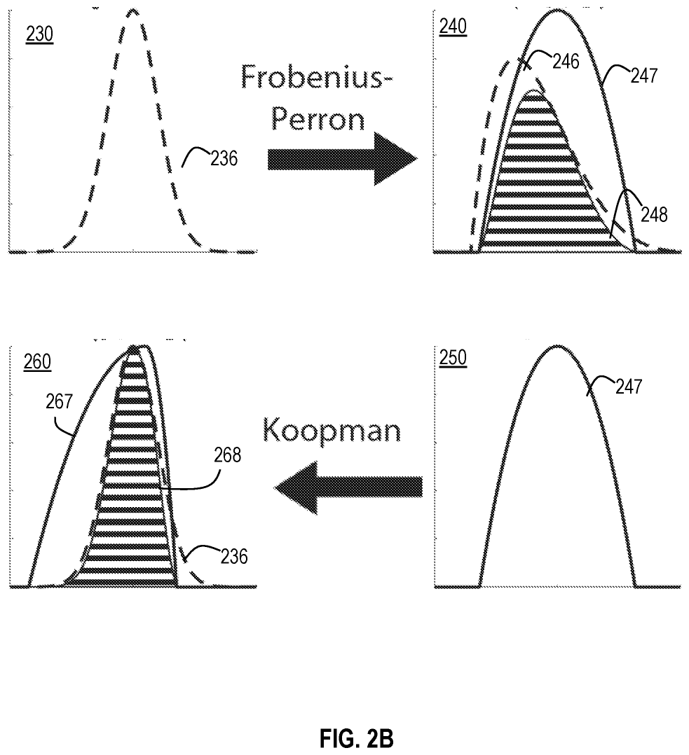

Here f is the posterior probability distribution given by the Bayesian estimation for a given initial condition x, A is an interval for resulting dynamical variables of the dynamical system S. and S⁻¹(A), called the counter-image of A, is the initial condition interval that corresponds to the results interval A. Equation 5 indicates that the probability integral over the counter image is equal to the probability integral over interval A of the FP operator P_{S} operating on the posterior probability distribution. Thus, P_{S} operating on the probability distribution of initial conditions gives the probability distribution of resulting states. Integrating the former over a results interval A gives the same probability as integrating the initial conditions over the counter image, S⁻¹(A). FIG. 2A is a pair of graphs that illustrate an example relationship between initial conditions probability density function (pdf) on graph 210 and resulting dynamic variable probability density (pdf) function on graph 220, according to an embodiment. In graph 210, the horizontal axis indicates initial condition values for all the parameters, e.g., initial conditions x₀; and, the vertical axis indicates probability density. The initial conditions pdf are given by trace 216. In graph 220, the horizontal axis indicates resulting dynamic variables values S; and, the vertical axis indicates probability density. The resulting dynamic variable values pdf are given by trace 226. Conditions x₀ that occur with probability f(x₀) results in dynamic variable S(x₀)=uᵢⱼ with initial condition x₀, with different probability, given by probability density distribution P_{S}f(x₀), because other initial conditions can add to or subtract from the occurrence of S(x₀). A range of initial conditions given by S⁻¹(A) results in range of dynamic variable values given by A. Because of the definition of P_{S} in Equation 5, the cross hatched areas under the two traces are equal.

In terms of dosing, the uncertainty in the population fixed and individual random effects, i.e. θ and η according to the probability distribution f(x), induces a probability distribution on the solution of the dynamics u(t), and this pushforward of the probability distribution through the dynamical system is the FP operator, i.e. [symbol] [P_{S}=x]= [symbol] [u(t, θ, η)=S(A)]. If S is both measurable and nonsingular, then P_{S} is uniquely defined by Equation 5. Monte Carlo estimation of probabilistic quantities of the dynamical system's solution thus correspond to approximating the pushed forward probability mass P_{S} f by direct sampling of initial conditions from the probability distribution f.

For the dosing problem, it would be useful to know the reverse operation for P_{S}, so that a specified range of values for u (or measurements y) can be given different weights using a cost function, and then the resulting cost function weighted results can be associated with a range of initial conditions, e.g., useful to determine a so-called push backward of the cost function weighted probability density function. Then a range of desirable results and a range of undesirable results can be specified when determining the initial dose to administer. In various embodiments, the Koopman operator is invoked instead of previous approaches to serve this purpose.

The Koopman operator, U_{S}, is defined by Equation 6.

U_{S}g(x)=g(S(x)) (6)

Here g is a function, such as a cost function, of the observed quantities y of the solution u to the dynamical system S. Note that U_{S} is adjoint to the FP operator, P_{S}, as given by Equation 7, where the symbols [symbol] a, b [symbol] represent the inner product of vectors a and b, In integral form

[symbol]

P_{S}f,g [symbol] =∫A g(x)PS f(x)μ(dx)=∫A U_{S}g(x)f(x)μ(dx),∀A∈ [symbol] = [symbol] f,U_{S}g [symbol] (7)

Equation 7 can be rewritten as Equation 8 showing the expectation form:

[symbol]

E[g(X)|X˜P_{S}f]=E[U_{S}g(X)|X˜f] (8)

where E[g(X)|X˜P_{S}f] means the expectation of g(X) where the X is a random variable distributed according to the probability distribution P_{S}f, and E[U_{S}g(X)|X˜f] means the expectation of U_{S}g(X) where X is the random variable distributed according to the probability distribution f[5]. Recall f is the pdf of the initial conditions vector x and that P_{S} f is the pdf of the resulting dynamic variable vector u. The left side of Equation 8 is referred to as the FP Expectation; and, the right side of Equation 8 is referred to as the Koopman Expectation. Equation 8 indicates that the probability of the results weighted by the cost function is equal to the probability of the initial conditions weighted by the Koopman operation on the cost function. The concepts of Equation 8 are depicted in FIG. 2B.

FIG. 2B is a set of plots that illustrates interaction of uncertainty in initial conditions represented by a pdf and a cost function using a Koopman adjunct process, according to an embodiment. In plot 230 and plot 260, the horizontal axis indicates the initial conditions x and the vertical axis is dimensionless, in a range between zero and one. The assumed known pdf of the initial conditions f(x) is illustrated by dashed trace 236. In plot 240 and 250, the horizontal axis indicates the resulting measured variable y; and the vertical axis is dimensionless, also in some range between zero and one, representing the probability density of y. In this space of plots 240 and 250, the cost function g(y) is illustrated by solid trace 247. The FP operator transforms the pdf of the initial conditions trace 236 to the results pdf illustrated by dashed trace 246 in plot 240. The dot product of the two, representing the left side of Equation 8, is given by the shaded area bounded by trace 248. This is the pdf for the cost function weighted results. The expected value, E[g(X)|X˜P_{S}f], is represented by the area of the shaded region. The Koopman operator U_{S} transforms the cost function g illustrated by trace 247 in plot 250 to its form in initial conditions space, U_{S} g, illustrated by trace 267 in plot 260. The dot product of U_{S} g and the initial conditions pdf f illustrated by trace 236, representing the right side of Equation 8, is given by the shaded area bounded by trace 268. This is the pdf for the cost function weighted initial conditions. The expected value, E[U_{S}g(X)|X˜f], is represented by the area of the shaded region. The inner products, represented by the area of the filled regions under traces 248 and 269, respectively, are equivalent.

Accounting for the uncertainty f(x) in initial conditions x propagation through a dynamical system S, the optimal dose is one which has the highest expectation of good patient outcomes. In other words, we wish to find the dosing schedule D which optimizes the expectation with respect to a cost function g on the solution u (or measured value y) of the dynamical system S. If we let g(u(t)) be the cost associated with a given patient outcome, this corresponds to finding a preferred dose regimen D* that satisfies Equation 9.

$${D}^{*} = \begin{matrix}arg min \\ D\end{matrix} E [ g ( X ) \vert X \sim {P}_{s} f ] \qquad ( 9 )$$

Corresponding to minimizing the KP Expectation. Equation 8 shows that Equation 9 is equivalent to minimizing the Koopman Expectation, as given by Equation 10.

$${D}^{*} = \begin{matrix}arg min \\ D\end{matrix} E [ {U}_{s} g ( X ) \vert X \sim f ] \qquad ( 10 )$$

Noting from FIG. 2B, the Koopman operator pulls back to the original initial condition probability distribution. Thus, Equation 10 can be evaluated using Equation 11.

E[U_{S}g(X)|X˜f]= [symbol] U_{S}g(x)f(x)dx (11)

The advantage of using Equation 11 can be understood as follows. The common way to perform dosing optimization with respect to parametric uncertainty expressed in Equation 9 is to utilize a Monte Carlo estimation of P_{S}f in order to evaluate the expectation. This involves: 1) Sample parameters θ and ηi from the uncertainty distribution f; 2). Solve the dynamical system to compute S(x) for each set of parameters; 3) Compute g(S(x)) on each set of parameters, and take the discrete average. This procedure is computationally expensive since it requires the solution of many differential equations. Using Equation 11, the desired expectation can be calculated much more efficiently, e.g., using a multidimensional quadrature where U_{S}g(x) is the solution of the dynamical system at parameters determined by the quadrature procedure and f(x) is the evaluation of the probability distribution at the quadrature points. The quadrature procedure is described in document Cubature_(Multi-dimensional integration), in subfolder index.php of folder wiki in subdomain initio of domain mit of super-domain edu.

Cost functions g are advantageously based on clinical trials used to establish safety guidelines known as the therapeutic window. For example, for a given drug the area under the curve (the total concentration or AUC) of the drug concentration in the target tissue or other dynamic variable u (or measurement y dependent on u) is a common quantity in which therapeutic windows are written. over 24 hour periods may be known to be safe when it is between 200 and 400 milligrams. Given how every individual metabolizes at given rates, the goal is to optimize the probability that the dose will be in the therapeutic window with respect to the uncertainty in the patient-specific effects. Any cost function g(u) may be used. In some embodiments, the cost function not only accounts for “goodness” in giving preferential weight (low cost) to results in the therapeutic window but also allows for cost of “badness,” such as avoiding certain ranges by assigning high cost to those ranges or increasing cost for distance from the therapeutic window. Using distance removed from the therapeutic window encapsulates the idea that being close to the therapeutic window might be sufficient while being far away incurs a greater risk on the patient.

To illustrate the method, an embodiment using a simple cost function is described here. Let g(y)=χw(y) where χw is an indicator function for the therapeutic window W, e.g., g is a function defined as 1 if the measured value y for the solution u to the differential equation with parameters (θ, η, D, Z) falls in the therapeutic window W, and 0 otherwise. With this definition of the cost function, the expectation of a characteristic function is the probability that the event occurs, and as such this definition of g gives rise to Equation 12.

$${D}^{*} = \begin{matrix}arg min \\ D\end{matrix} P [ y ( t ) ∈ W ] \qquad ( 12 )$$

Therefore, under this choice of g the optimal dosing regimen D* computed by the optimization over the Koopman expectation is the dosing regimen that has the maximal probability of the patient's outcomes to be in the therapeutic window.

Numerical methods for computing integral expressions like in Equation 11 are called quadrature methods. To compute the quadrature, the method can use several efficient computational approaches. In embodiments in which the dimensionality of the integral (the number of elements in the vector x of initial values) is sufficiently small, then techniques like Cubature can be effectively used. However, in other embodiments in which the dimensionality of the parameter space is higher, a dimension-independent quadrature method, such as Monte Carlo importance sampling methods like VEGAS can be used to increase efficiency of calculation. While the solution of a naive Monte Carlo approaches the expectation with an error proportional to O(√N), where N is the dimensionality of the integrand, due to the Central Limit Theorem, these importance sampling techniques can allow for dramatically increased convergence rates such as O(N) for VEGAS.

Because each integrand calculation requires exactly one solution of the dynamical system, which is the most expensive portion of the calculation (usually a numerical solution to an ODE), this reformulation can dramatically reduce the computational cost of calculating the cost function. In addition, each of these quadrature methods allow for a high degree of parallelism. Implementations like Julia program Cuba.jl, available from folder cuba at domain feynarts of super-domain de, and Cubature.jl, available from, folder ˜stevenj at subdomain math of domain mit of super-domain edu, allow for user-chosen batch sizes M of simultaneous integrand calculations.

In one embodiment, a Julia language tool, such as DiffEqGPU.jl, can be invoked to use nonstandard interpretation to automatically generate data-parallel GPU-parallelized ODE solvers from any existing pure-Julia ODE solver code with generic AbstractArray handling. This metaprogram-generated ODE integrator simultaneously computes the solution of an ODE for different parameters, splitting the computation amongst the many cores of the GPU, and the solution is then used to calculate the loss at all M parameters. Because it applies to any existing pure Julia ODE solver, this method generates GPU-based integrators for both non-stiff and stiff ODEs, including implicit-explicit (IMEX) methods, and adaptive methods for stochastic differential equations (SDEs), delay differential equations (DDEs), differential algebraic equations (DAEs), hybrid differential equations such as jump diffusion or Markov regime switching models, and more, totaling over 200 methods. The loss of each of the M trajectories is then weighted by the probability of the parameters to finish the computation of the integrand.

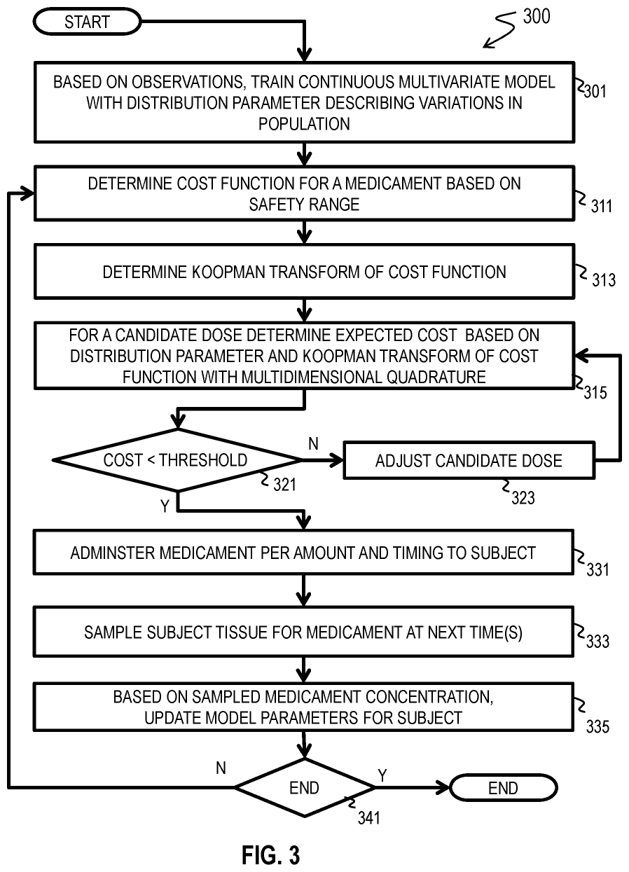

FIG. 3 is flow diagram that illustrates an example method for determining a dose that will minimize a cost, according to an embodiment. Although steps are depicted in FIG. 3 as integral steps in a particular order for purposes of illustration, in other embodiments, one or more steps, or portions thereof, are performed in a different order, or overlapping in time, in series or in parallel, or are omitted, or one or more additional steps are added, or the method is changed in some combination of ways.

In step 301, a NLME model is developed with appropriate random and nonrandom parameters; and, the model parameters are learned based, for example, on historical data in the EHR. The training continues until a certain stop condition is achieved, such as a number of iterations, or a maximum or percentile difference between model output and measured values is less than some tolerance, or a difference in results between successive iterations is less than some target tolerance, or the results begin to diverge, or other known measure, or some combination. As a result of the training, the model is a continuous multivariate model that requires no classification or binning of the subject be performed, as is required in the application of the nomograms of the prior art. The model includes at least one parameter based on population average and at least one parameter (e.g., Ω) describing random deviations among individuals, as well as at least one portion describing dynamics (e.g., using ordinary differential equations describing changes over time). As a result of step 301, a continuous multivariate model is received on a processor as first data that indicates, for dose response to a medicament, with at least one distribution parameter.

In step 311 a cost function is determined based on a safety range, such as a therapeutic range or a therapeutic index or some combination. In some embodiments, the cost function includes a weighted function of distance from the therapeutic range. For example, there is no cost for a result within the therapeutic range, and an increasing cost with increasing distance above the therapeutic range. That can be linear or have any shape function with distance. In some embodiments, an especially toxic range, e.g., as determined based on the therapeutic index, is given a higher weight that is not necessarily proportional to distance from the therapeutic range. In embodiments implemented automatically on a processor, step 311 includes: receiving second data that indicates a therapeutic range of values for the dose response; and, receiving third data that indicates a cost function based on distance of a dose response from the therapeutic range.

In step 313 a Koopman transform for the cost function is determined or programmed on the processor by defining a numerical procedure to calculate g(S(x)), e.g., defining a function that takes in an initial condition x₀ and parameters of the population, including the distribution parameters, runs the dynamics, and evaluates the cost.

In step 315, for a candidate dose, the expected cost of that candidate dose is determined based on the at least on parameter of random deviations (e.g., Ω for each of one or more model parameters) and the Koopman transform of the cost function using multidimensional quadrature methods. In some of these embodiment the evaluation is performed, e.g., based on known graphical processing unit acceleration techniques.

In step 321, it is determined whether the cost is less than some target threshold cost, such as a cost found to associated with good outcomes. If not, control passes to step 323 to adjust the candidate dose, e.g., by reducing the candidate dose an incremental amount or percentage. Control then passes back to step 315 to recompute the cost. If the evaluated cost is less than the threshold, then control passes to step 331.

In step 331 the medicament is administered to the subject per the candidate dose. Thus, when the expected cost is less than a threshold cost, the candidate dose of the medicament is caused to be administered to a subject.

In step 333, a sample is taken from the subject to determine therapeutic effect (e.g., concentration of the medicament in the tissue of the sample) at the zero or more measurement times determined during step 325. This step is performed in a timely way to correct any deficiencies in administering the medicament before harmful effects accrue to the subject, such as ineffective or toxic results. This step is in contrast to previous methods, in which the next dose of the medicament is administered regardless of whether the first dose was ineffective or toxic for the individual subject. Thus, step 327 includes sampling the subject at the set of one or more times to obtain corresponding values of the corresponding measures of the therapeutic effect.

In step 335 the model determined in step 305 is updated, if at all, based on the measurements taken in step 333. For example, if the medicament concentration value is less than or greater than the therapeutic range, it is determined that the individual has a response that deviates substantively from the population average values. Any method may be used to update the model parameters to fit the observed response of the individual, such as new training of the neural network. In some embodiments, the population model is updated based on the one observation of the individual being treated in proportion to the number of different subjects that previously contributed to the model. In some embodiments, in addition to or instead of updating the population model parameter values, special subject-specific model parameter values are generated.

The method 300 thus determines, during treatment of a subject, individualized protocols for administering medicaments, which have increased chance to be maintained in the subject within a therapeutic range. The extra formalism is useful because of the tremendous performance and feature benefits

As personalized precision dosing becomes more commonplace, efficient and robust methods for optimizing dosing with respect to probabilistic outcomes will become more central to pharmacometric practice. As personalized computations become pervasive, more accurate models will be required, and the computations will need to migrate to the patient, living on embedded or mobile devices with little compute power.

### 2. EXAMPLE EMBODIMENTS

In various example embodiments, dosing is determined using some or all the steps of method 300 for medicaments listed in the following tables. For each medicament listed, a concentration window W or therapeutic index is listed in a third column. The therapeutic index (TI, also referred to as therapeutic ratio) is a quantitative measurement of the relative safety of a drug. It is a comparison of the amount of a therapeutic agent that causes the therapeutic effect to the amount that causes toxicity. As proposed herein, a cost function g(y) is based, at least in part, on a value in this third column. The Koopman Expectation is used in Equation 11 or Equation 12 with a quadrature procedure to deduce an optimum range or subset of the dosing range listed in the second column.

TABLE 1
ANTI-EPILEPTICS:
* Uncertain reference range ; ** Well defined
toxic range; NE = Not Established ;

| | Dose range | Concentration range |
|---|---|---|
| Phenytoin (PHE) | (non-linear PK) | (10-20 mg/L) |
| Carbamazepine | (800-1600 mg) | (4-8/12 mg/L) |
| (CBZ) | | |
| Valproic acid (VPA) | (non-linear PK) | (50-100 mg/L) |
| Phenobarbital (PHB) | (100-600 mg) | (10-40 mg/L) |
| Primidone | (750-2000 mg) | (5-10 mg/L) |
| Clonazepam | (8-20 mg) | (0.02-0.07 mg/L) |
| Clobazam | (20-80 mg) | (0.03-0.3 mg/L) |
| Ethosuximide | (500-1500 mg) | (40-100 mg/L) |
| Ezogabine | (600-1200 mg) | NE |
| Felbamate | (1200-3600 mg) | (30-60 mg/L)* |
| Gabapentin | (900-2400 mg) | (2-20 mg/L) |
| Lacosamide | (300-400 mg) | (5-15 mg/L) |
| Lamotrigine | (300-500 mg) | (3-14 mg/L) |
| Levetiracetam | (1000-3000 mg) | (12-46 mg/L) |
| Oxcarbazepine | (1200-2400 mg) | (3-35 mg/L)** |
| Perampanel | (8-12 mg) | NE |
| Pregabalin | (300-600 mg) | (2-5/8 mg/L) |
| Rufinamide | (1600-3200 mg) | (30-40 mg/L)* |
| Tiagabine | (30-56 mg) | (0.02-0.2 mg/L)* |
| Topiramate | (100-400 mg) | (5-20 mg/L) |
| Vigabatrin | (2000-3000 mg) | (0.8-36 mg/L) |
| Zonisamide | (100-600 mg) | (10-40 mg/L)** |

‡ therapeutic index (TI) = ratio between upper/lower bounds of therapeutic window (TW)

TABLE 2
ANTICOAGULANTS

| | Dose range | Concentration/biomarker range |
|---|---|---|
| Argatroban | NA | (1.5-3 × aPTT) |
| Dabigatran | NA | (60-200 mg/L or |
| | | 1.5-2 × base aPTT) |
| Warfarin | (2-10 mg | (INR: 2-3) |
| | Generally starts | |
| | at 5 mg) | |

TABLE 3
ANTIARRHYTHMICS

| | Dose range | Concentration/biomarker range |
|---|---|---|
| Amiodarone | (200-400 mg) | |
| Digoxin | (0.125-0.375 mg | (0.8-2: AF |
| | up to 0.5 mg QD) | 0.5-1: Cl) |
| Flecainide | (50-150/200 mg BID) | (200-1000 ng/mL ?) |
| Quinidine | (200 mg-400 mg × | (2-5 mg/L) |
| | 4/day) | |
| Sotalol | (80-160 mg BID) | NA |

TABLE 4
IMMUNOSUPPRESSANTS

| | Small Separation | Small change in |
|---|---|---|
| | therapeutic/toxic | concentration may lead to |
| | doses | failure/serious AE ‡ |
| Cyclosporine | (3-6 mg/kg BID) | (100-200 ng/mL) |
| Everolimus | (Initial: 0.75 mg BID) | (3-8 ng/mL) |
| Mycophenolate | 1000 mg-1500 | (30-60 mg*h/L |
| | mg BID | not in label) |
| | (different transplant) | |
| Sirolimus | 0 | (12-20 ng/mL) |
| | (Initial: 2-5 mg QD | |
| | With-without CsA) | |
| Tacrolimus | (Initial: 0.1 to 0.2 | (5-15 ng/mL) |
| | mg/kg/day) | |

TABLE 5
ANTICOAGULANTS:

| Drug | Therapeutic window | Therapeutic index |
|---|---|---|
| Argatroban | 2-3 × baseline aPTT | 1.7 |
| | (60-100 seconds) | |
| Dabigatran | 2-3 × baseline aPTT | 1.7 |
| | (60-100 seconds) | |
| Warfarin | INR: 2-3 | 1.5 |

TABLE 6
ANTIARRHYTHMICS:

| Drug | Therapeutic window | | Therapeutic index |
|---|---|---|---|
| Digoxin | 0.5-2 | ng/mL | 4 |
| Flecainide | 0.2-1 | mg/L | 5 |
| Quinidine | 2-5 | mg/L | 2.5 |

TABLE 7
ANTIEPILEPTICS:

| Drug | Therapeutic window | Therapeutic index |
|---|---|---|
| Carbamazepine | 4-12 mg/L | 3 |
| Felbamate | 30-60 mg/L | 2 |
| Lamotrigine | 3-14 mg/L | 4.7 |
| Oxcarbazepine | 3-35 mg/L | 11.7 |
| Phenobarbital | 10-40 mg/L | 4 |
| Phenytoin | 10-20 mg/L | 2 |
| Tiagabine | 0.02-0.2 mg/L | 10 |
| Topiramate | 5-20 mg/L | 4 |
| Valproate | 50-100 mg/L | 2 |
| Zonisamide | 10-40 mg/L | 4 |

According to an example embodiment, at least a portion of the method 300 is implemented for determining and administering a dose regimen for Theophylline to deliver and maintain a blood concentration within a therapeutic range. In this embodiment is demonstrated how utilizing the Koopman expectation can accelerate the computation 100× over Monte Carlo while giving numerical error bounds for improved clarity of the results. This methodology is implemented with the high performance differential equation solvers of DifferentialEquations.jl within the Pumas pharmacometrics suite so that existing models can automatically be compatible with the accelerated computation. Being a pure Julia software stack, this methodology can compile to mobile devices like ARM for direct deployment to patients.

In this example embodiment, a high-performance implementation of the Koopman Expectation in Pumas. Pumas is a high-performance pharmaceutical modeling and simulation engine written in the Julia Language. At its core, Pumas uses the DifferentialEquations.jl ecosystem to drive its event based models that can be used for characterizing time course of drugs or other endogenous substances in the human body. Such characterization in Pumas facilitates accurate dosing predictions and optimization of treatment trajectories. Programs were developed in the Julia programming (at domain julialang super-domain org). Equation 11 was implemented by utilizing DifferentialEquations.jl [3] to calculate U_{S}g given a Pumas specification of a dynamical system S. In other embodiments, alternative ODE solvers for the dynamical equations, such as Sundials (available at subdomain computing of domain llnl of super-domain gov in folder projects subfolder sundails.gov subfolder projects document sundials), or LSODA (available at subdomain people of subdomain sc of subdomainfsu of super-domain edu in folder ˜jburkardt subfolderf77_src subfolder odepack document odepack.html), could also be used to evaluate the dynamics. The multidimensional integral was calculated using Quadrature.jl, a wrapper library over common quadrature methods such as Cuba and Cubature, referenced above. This integration implementation allows for a batch solve that parallelizes the computation over the quadrature points, allowing for multithreaded, distributed, and GPU acceleration of the quadrature.

Table 8 lists code that demonstrates the use of the Koopman expectation for the calculation of the probability that the AUC will be below 300 on the Theophylline model. The nonlinear mixed effect model's definition is defined using Pumas' standard @model macro form. In this example, there are no individual random effects η, the @param block defines the fixed effects θ as theta and denotes that it is a vector of 4 variables. The second blog @covariates defines the covariates as Zᵢ=[sexᵢ, wtᵢ, etnᵢ] where the specific values for subject i are pulled from patient data. The pre block determines the values as used in the dynamic system [K aᵢ, CLᵢ, Vᵢ] as a function of the covariates, fixed effects, and random effects. Note in this example the random effects ηᵢ are omitted. @dynmics defines the dynamical equation \dot{u}ᵢ=f(uᵢ, Dᵢ, ηᵢ, θ, Zᵢ) where uᵢ(t)=[Depotᵢ(t), Centralᵢ(t)]. @derived defines the computation of the output variables from the dynamical variables, i.e. a computational specification of ψ(uᵢⱼ) the timeseries cpᵢ(t)=Centralᵢ/Vᵢ is calculated, from which the area under the curve is calculated via standard noncompartmental analysis (NCA) procedures. In total, this is a computational specification of a specific (θ, Zᵢ, Ω, Π, Σ)→yᵢⱼ=yᵢ (tᵢⱼ) map from Equation (1d) for the Theophylline model.

TABLE 8

Mixed model definition for Theophylline.
model = @model begin
@param begin
theta in VectorDomain(4)
end
@covariates sex wt etn
@pre begin
Ka = theta [1]
CL = theta[2] * ((wt/70)\hat{ }0.75) * (theta[4]\hat{ }sex)
V = theta[3]
end
@dynamics begin
Depot′ = −Ka*Depot
Central′ = Ka*Depot − CL* cp
end
@derived begin
cp = Central ./ V
nca := @nca cp
auc = NCA.auc(nca)
end
end

Then to define the subject and the dosage regimen, we read in a dataset of values for covariates Z. Next we input the parameter uncertainty distributions for θ and ηᵢ. This would normally come from Bayesian posteriors as described above. but for purposes of illustration a prescribe distribution is specified. In our demonstration we used θ˜[Uniform(1.0,5.0), Uniform(20.0,100.0), Uniform(200.0,400.0), Uniform(0.1,2.0)], meaning θ is a 4-dimensional random variable where its first value is defined by the uniform random variable on Uniform(1.0,5.0), and similar for the other values. The probability distribution of Uniform(a, b) is f(x)=1/(b−a) when x∈[a, b], otherwise it is zero. We then define the therapeutic window via the loss function g on the observables. For this implementation, we used the Pumas NLME integration with NCA to compute the AUC as part of the derived variables from the model, and thus the AUC exists as one of the observed outputs of the system. We can thus define the therapeutic window as less than a threshold value. Now one calls the expectation routine and tell it to use the Koopman Expectation method. In there we designate that we would like to use the HCubatureJL quadrature method, which tends to be efficient for low dimensional integrands (<8 uncertain variables). This computation then computes the given expectation to the desired tolerance. Given its differentiability, this function can then be used inside of an optimization loop with defined dosage regimens to thus perform dosage optimization under uncertainty.

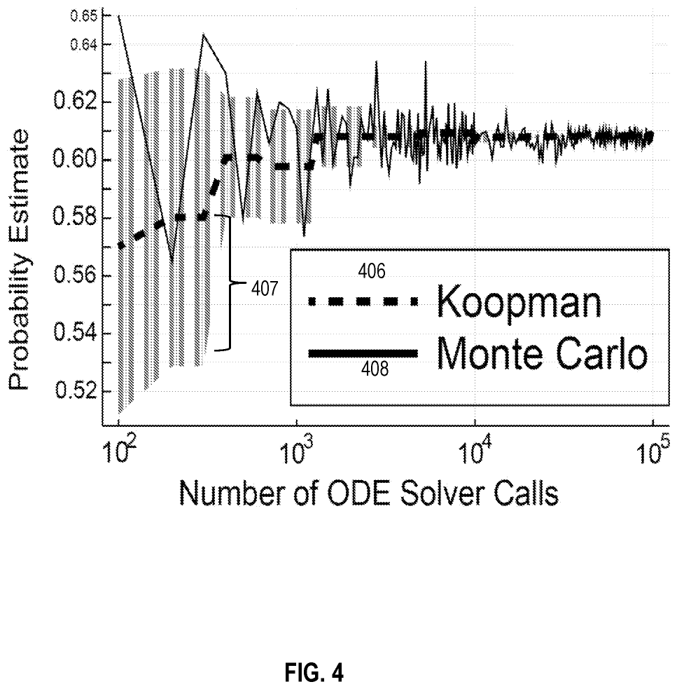

To demonstrate the tremendous performance improvement and feature benefits, we compared the KoopmanExpectation option to the MonteCarloExpectation option with various increasing choices of imaxiters (allowed number of ODE solves) to determine the rate of convergence of the two methods for calculating the probability of patient outcomes occurring in the therapeutic window. FIG. 4 is a graph that illustrates example convergence of the Koopman Expectation calculation compared to a previous method using Monte Carlo Expectation, according to an example embodiment. The horizontal axis indicates the number of calls to an ODE solver, an indication of computational cost; and, the vertical axis indicates the probability estimate. Here the Koopman Expectation was evaluated using the routine Cubature on the integral shown as trace 406. The convergence of the Monte Carlo is shown as trace 408. Both are given in terms of the number of ODE solver calls required. Note that the cubature integration method trace 408 comes with a free error estimation indicated by bounds 407.

FIG. 4 demonstrates two results. One result is that the Koopman method converges to give stable probability estimates of the second digit with nearly 100× fewer ODE solves, resulting in roughly two orders of magnitude less computational cost. A second result is that, by utilizing the HCubature method, the Koopman calculation not only determines the probability but also gives numerical error bounds on the probability estimate, allowing the user to effectively know the uncertainty introduced by the numerical error. Such a bound is highly difficult to calculate with Monte Carlo estimates given the slow rate of convergence of the variance. Together, this demonstrates the Koopman expectation as both a method for efficiency and robustness.

### 3. COMPUTATIONAL HARDWARE OVERVIEW

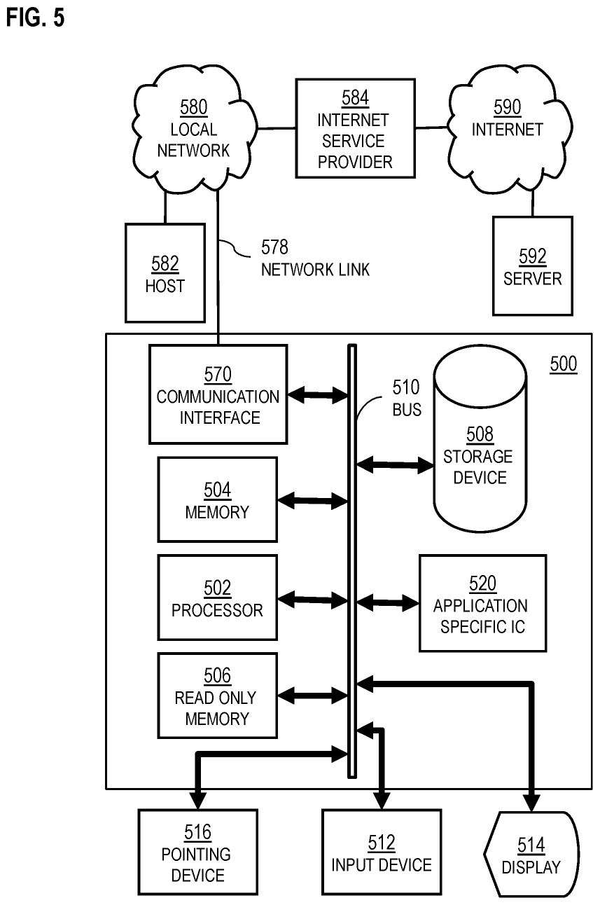

FIG. 5 is a block diagram that illustrates a computer system 500 upon which an embodiment of the invention may be implemented. Computer system 500 includes a communication mechanism such as a bus 510 for passing information between other internal and external components of the computer system 500. Information is represented as physical signals of a measurable phenomenon, typically electric voltages, but including, in other embodiments, such phenomena as magnetic, electromagnetic, pressure, chemical, molecular atomic and quantum interactions. For example, north and south magnetic fields, or a zero and non-zero electric voltage, represent two states (0, 1) of a binary digit (bit). Other phenomena can represent digits of a higher base. A superposition of multiple simultaneous quantum states before measurement represents a quantum bit (qubit). A sequence of one or more digits constitutes digital data that is used to represent a number or code for a character. In some embodiments, information called analog data is represented by a near continuum of measurable values within a particular range. Computer system 500, or a portion thereof, constitutes a means for performing one or more steps of one or more methods described herein.

A sequence of binary digits constitutes digital data that is used to represent a number or code for a character. A bus 510 includes many parallel conductors of information so that information is transferred quickly among devices coupled to the bus 510. One or more processors 502 for processing information are coupled with the bus 510. A processor 502 performs a set of operations on information. The set of operations include bringing information in from the bus 510 and placing information on the bus 510. The set of operations also typically include comparing two or more units of information, shifting positions of units of information, and combining two or more units of information, such as by addition or multiplication. A sequence of operations to be executed by the processor 502 constitutes computer instructions.

Computer system 500 also includes a memory 504 coupled to bus 510. The memory 504, such as a random access memory (RAM) or other dynamic storage device, stores information including computer instructions. Dynamic memory allows information stored therein to be changed by the computer system 500. RAM allows a unit of information stored at a location called a memory address to be stored and retrieved independently of information at neighboring addresses. The memory 504 is also used by the processor 502 to store temporary values during execution of computer instructions. The computer system 500 also includes a read only memory (ROM) 506 or other static storage device coupled to the bus 510 for storing static information, including instructions, that is not changed by the computer system 500. Also coupled to bus 510 is a non-volatile (persistent) storage device 508, such as a magnetic disk or optical disk, for storing information, including instructions, that persists even when the computer system 500 is turned off or otherwise loses power.

Information, including instructions, is provided to the bus 510 for use by the processor from an external input device 512, such as a keyboard containing alphanumeric keys operated by a human user, or a sensor. A sensor detects conditions in its vicinity and transforms those detections into signals compatible with the signals used to represent information in computer system 500. Other external devices coupled to bus 510, used primarily for interacting with humans, include a display device 514, such as a cathode ray tube (CRT) or a liquid crystal display (LCD), for presenting images, and a pointing device 516, such as a mouse or a trackball or cursor direction keys, for controlling a position of a small cursor image presented on the display 514 and issuing commands associated with graphical elements presented on the display 514.

In the illustrated embodiment, special purpose hardware, such as an application specific integrated circuit (IC) 520, is coupled to bus 510. The special purpose hardware is configured to perform operations not performed by processor 502 quickly enough for special purposes. Examples of application specific ICs include graphics accelerator cards for generating images for display 514, cryptographic boards for encrypting and decrypting messages sent over a network, speech recognition, and interfaces to special external devices, such as robotic arms and medical scanning equipment that repeatedly perform some complex sequence of operations that are more efficiently implemented in hardware.

Computer system 500 also includes one or more instances of a communications interface 570 coupled to bus 510. Communication interface 570 provides a two-way communication coupling to a variety of external devices that operate with their own processors, such as printers, scanners and external disks. In general the coupling is with a network link 578 that is connected to a local network 580 to which a variety of external devices with their own processors are connected. For example, communication interface 570 may be a parallel port or a serial port or a universal serial bus (USB) port on a personal computer. In some embodiments, communications interface 570 is an integrated services digital network (ISDN) card or a digital subscriber line (DSL) card or a telephone modem that provides an information communication connection to a corresponding type of telephone line. In some embodiments, a communication interface 570 is a cable modem that converts signals on bus 510 into signals for a communication connection over a coaxial cable or into optical signals for a communication connection over a fiber optic cable. As another example, communications interface 570 may be a local area network (LAN) card to provide a data communication connection to a compatible LAN, such as Ethernet. Wireless links may also be implemented. Carrier waves, such as acoustic waves and electromagnetic waves, including radio, optical and infrared waves travel through space without wires or cables. Signals include man-made variations in amplitude, frequency, phase, polarization or other physical properties of carrier waves. For wireless links, the communications interface 570 sends and receives electrical, acoustic or electromagnetic signals, including infrared and optical signals, that carry information streams, such as digital data.

The term computer-readable medium is used herein to refer to any medium that participates in providing information to processor 502, including instructions for execution. Such a medium may take many forms, including, but not limited to, non-volatile media, volatile media and transmission media. Non-volatile media include, for example, optical or magnetic disks, such as storage device 508. Volatile media include, for example, dynamic memory 504. Transmission media include, for example, coaxial cables, copper wire, fiber optic cables, and waves that travel through space without wires or cables, such as acoustic waves and electromagnetic waves, including radio, optical and infrared waves. The term computer-readable storage medium is used herein to refer to any medium that participates in providing information to processor 502, except for transmission media.

Common forms of computer-readable media include, for example, a floppy disk, a flexible disk, a hard disk, a magnetic tape, or any other magnetic medium, a compact disk ROM (CD-ROM), a digital video disk (DVD) or any other optical medium, punch cards, paper tape, or any other physical medium with patterns of holes, a RAM, a programmable ROM (PROM), an erasable PROM (EPROM), a FLASH-EPROM, or any other memory chip or cartridge, a carrier wave, or any other medium from which a computer can read. The term non-transitory computer-readable storage medium is used herein to refer to any medium that participates in providing information to processor 502, except for carrier waves and other signals.

Logic encoded in one or more tangible media includes one or both of processor instructions on a computer-readable storage media and special purpose hardware, such as ASIC 520.

Network link 578 typically provides information communication through one or more networks to other devices that use or process the information. For example, network link 578 may provide a connection through local network 580 to a host computer 582 or to equipment 584 operated by an Internet Service Provider (ISP). ISP equipment 584 in turn provides data communication services through the public, world-wide packet-switching communication network of networks now commonly referred to as the Internet 590. A computer called a server 592 connected to the Internet provides a service in response to information received over the Internet. For example, server 592 provides information representing video data for presentation at display 514.

The invention is related to the use of computer system 500 for implementing the techniques described herein. According to one embodiment of the invention, those techniques are performed by computer system 500 in response to processor 502 executing one or more sequences of one or more instructions contained in memory 504. Such instructions, also called software and program code, may be read into memory 504 from another computer-readable medium such as storage device 508. Execution of the sequences of instructions contained in memory 504 causes processor 502 to perform the method steps described herein. In alternative embodiments, hardware, such as application specific integrated circuit 520, may be used in place of or in combination with software to implement the invention. Thus, embodiments of the invention are not limited to any specific combination of hardware and software.

The signals transmitted over network link 578 and other networks through communications interface 570, carry information to and from computer system 500. Computer system 500 can send and receive information, including program code, through the networks 580, 590 among others, through network link 578 and communications interface 570. In an example using the Internet 590, a server 592 transmits program code for a particular application, requested by a message sent from computer 500, through Internet 590, ISP equipment 584, local network 580 and communications interface 570. The received code may be executed by processor 502 as it is received, or may be stored in storage device 508 or other non-volatile storage for later execution, or both. In this manner, computer system 500 may obtain application program code in the form of a signal on a carrier wave.

Various forms of computer readable media may be involved in carrying one or more sequence of instructions or data or both to processor 502 for execution. For example, instructions and data may initially be carried on a magnetic disk of a remote computer such as host 582. The remote computer loads the instructions and data into its dynamic memory and sends the instructions and data over a telephone line using a modem. A modem local to the computer system 500 receives the instructions and data on a telephone line and uses an infra-red transmitter to convert the instructions and data to a signal on an infra-red a carrier wave serving as the network link 578. An infrared detector serving as communications interface 570 receives the instructions and data carried in the infrared signal and places information representing the instructions and data onto bus 510. Bus 510 carries the information to memory 504 from which processor 502 retrieves and executes the instructions using some of the data sent with the instructions. The instructions and data received in memory 504 may optionally be stored on storage device 508, either before or after execution by the processor 502.

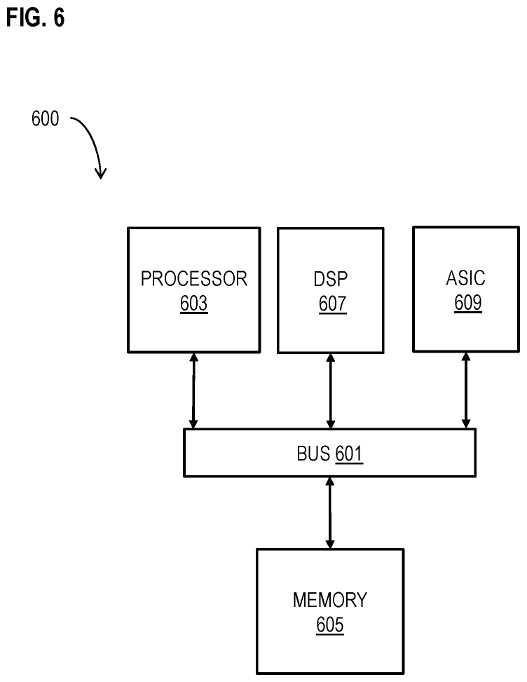

FIG. 6 illustrates a chip set 600 upon which an embodiment of the invention may be implemented. Chip set 600 is programmed to perform one or more steps of a method described herein and includes, for instance, the processor and memory components described with respect to FIG. 5 incorporated in one or more physical packages (e.g., chips). By way of example, a physical package includes an arrangement of one or more materials, components, and/or wires on a structural assembly (e.g., a baseboard) to provide one or more characteristics such as physical strength, conservation of size, and/or limitation of electrical interaction. It is contemplated that in certain embodiments the chip set can be implemented in a single chip. Chip set 600, or a portion thereof, constitutes a means for performing one or more steps of a method described herein.

In one embodiment, the chip set 600 includes a communication mechanism such as a bus 601 for passing information among the components of the chip set 600. A processor 603 has connectivity to the bus 601 to execute instructions and process information stored in, for example, a memory 605. The processor 603 may include one or more processing cores with each core configured to perform independently. A multi-core processor enables multiprocessing within a single physical package. Examples of a multi-core processor include two, four, eight, or greater numbers of processing cores. Alternatively, or in addition, the processor 603 may include one or more microprocessors configured in tandem via the bus 601 to enable independent execution of instructions, pipelining, and multithreading. The processor 603 may also be accompanied with one or more specialized components to perform certain processing functions and tasks such as one or more digital signal processors (DSP) 607, or one or more application-specific integrated circuits (ASIC) 609. A DSP 607 typically is configured to process real-world signals (e.g., sound) in real time independently of the processor 603. Similarly, an ASIC 609 can be configured to performed specialized functions not easily performed by a general purposed processor. Other specialized components to aid in performing the inventive functions described herein include one or more field programmable gate arrays (FPGA) (not shown), one or more controllers (not shown), or one or more other special-purpose computer chips.

The processor 603 and accompanying components have connectivity to the memory 605 via the bus 601. The memory 605 includes both dynamic memory (e.g., RAM, magnetic disk, writable optical disk, etc.) and static memory (e.g., ROM, CD-ROM, etc.) for storing executable instructions that when executed perform one or more steps of a method described herein. The memory 605 also stores the data associated with or generated by the execution of one or more steps of the methods described herein.

### 4. ALTERNATIVES, DEVIATIONS AND MODIFICATIONS

In the foregoing specification, the invention has been described with reference to specific embodiments thereof. It will, however, be evident that various modifications and changes may be made thereto without departing from the broader spirit and scope of the invention. The specification and drawings are, accordingly, to be regarded in an illustrative rather than a restrictive sense. Throughout this specification and the claims, unless the context requires otherwise, the word “comprise” and its variations, such as “comprises” and “comprising,” will be understood to imply the inclusion of a stated item, element or step or group of items, elements or steps but not the exclusion of any other item, element or step or group of items, elements or steps. Furthermore, the indefinite article “a” or “an” is meant to indicate one or more of the item, element or step modified by the article.

### 5. REFERENCES

The following citations are referenced in the above.
[1] Andrzej Lasota and Michael C. Mackey. Chaos, Fractals, and Noise: Stochastic Aspects of Dynamics. Springer Science & Business Media, Nov. 27, 2013. 481 pp. isbn: 978-1-4612-4286-4.
[2] Andrew Leonard. “Probabilistic Methods for Decision Making in Precision Airdrop”. GA Tech, Jan. 15, 2019. 141 pp.
[3] Christopher Rackauckas and Qing Nie. “DifferentialEquations.Jl—A Performant and Feature-Rich Ecosystem for Solving Differential Equations in Julia”. In: J. Open Res. Softw. 5.1 (1 May 25, 2017), p. 15. issn: 2049-9647. doi: 10.5334/jors.151.
[4] Andrea Yeong Weiße. “Global Sensitivity Analysis of Ordinary Differential Equations: Adaptive Density Propagation Using Approximate Approximations”. In: (2009). Accepted: 2018-06-07T22:43:56Z. at subdomain dx of domain doi of super-domain org in folder 10.17169 and file refubium-13784.
[5] Gerlach, A. R., Leonard, A., Rogers, J., & Rackauckas, C. (2020) “The Koopman Expectation: An Operator Theoretic Method for Efficient Analysis and Optimization of Uncertain Hybrid Dynamical Systems,” available at domain arxiv of super-domain org in folder abs at link 2008.08737v1 to file 2008.08737v1.pdf.

What is claimed is:

1. A method executed automatically on a processor for generating a dosing protocol for an individual, the method comprising:
receiving first data that indicates, for a dose response to a medicament, a continuous multivariate model of a population with at least one distribution parameter characterizing variations in the population;
receiving second data that indicates a therapeutic range of values for the dose response;
receiving third data that indicates a cost function based on distance of the dose response from the therapeutic range;
evaluating for a candidate dose an expected cost based at least in part on the distribution parameter and a Koopman transform of the cost function; and
when the expected cost is less than a threshold cost, causing the candidate dose of the medicament to be administered to a subject.

2. The method of claim 1, said evaluating the expected cost further comprising performing multidimensional quadrature.

3. The method of claim 1, wherein the at least one distribution parameter is based on a distribution of values in electronic health records (EHR).

4. The method of claim 1, wherein the medicament is a type selected from a group comprising anti-epileptics, anti-coagulants, anti-arrhythmics, and immunosuppressants.

5. The method of claim 1, wherein the distance of a dose response from the therapeutic range for the cost function is indicated by a therapeutic index.

6. A non-transitory computer-readable medium carrying one or more sequences of instructions for generating a dosing protocol for an individual, wherein execution of the one or more sequences of instructions by one or more processors causes the one or more processors to perform the steps of:
receiving first data that indicates, for a dose response to a medicament, a continuous multivariate model of a population with at least one distribution parameter characterizing variations in the population;
receiving second data that indicates a therapeutic range of values for the dose response;
receiving third data that indicates a cost function based on distance of the dose response from the therapeutic range;
evaluating for a candidate dose an expected cost based at least in part on the distribution parameter and a Koopman transform of the cost function; and
causing the candidate dose of the medicament to be administered to a subject.

7. The computer-readable medium of claim 6, said evaluating the expected cost further comprising performing multidimensional quadrature.

8. The computer-readable medium of claim 6, wherein the at least one distribution parameter is based on a distribution of values in electronic health records (EHR).

9. The computer-readable medium of claim 6, wherein the medicament is a type selected from a group comprising anti-epileptics, anti-coagulants, anti-arrhythmics, and immunosuppressants.

10. The computer-readable medium of claim 6, wherein the distance of a dose response from the therapeutic range for the cost function is indicated by a therapeutic index.

11. An apparatus for generating a dosing protocol for an individual, the apparatus comprising:
at least one processor; and
at least one memory including one or more sequences of instructions,
the at least one memory and the one or more sequences of instructions configured to, with the at least one processor, cause the apparatus to perform at least the following:
receive first data that indicates, for a dose response to a medicament, a continuous multivariate model of a population with at least one distribution parameter characterizing variations in the population;
receive second data that indicates a therapeutic range of values for the dose response;
receive third data that indicates a cost function based on distance of the dose response from the therapeutic range;
evaluate for a candidate dose an expected cost based at least in part on the distribution parameter and a Koopman transform of the cost function; and
when the expected cost is less than a threshold cost, cause the candidate dose of the medicament to be administered to a subject.

12. The apparatus of claim 11, said evaluating the expected cost further comprising performing multidimensional quadrature.

13. The apparatus of claim 11, wherein the at least one distribution parameter is based on a distribution of values in electronic health records (EHR).

14. The apparatus of claim 11, wherein the medicament is a type selected from a group comprising anti-epileptics, anti-coagulants, anti-arrhythmics, and immunosuppressants.

15. The apparatus of claim 11, wherein the distance of a dose response from the therapeutic range for the cost function is indicated by a therapeutic index.
